(19) United States
(12) Patent Application Publication
(10) Pub. No.: US 2022/0096324 A1
(43) Pub. Date: Mar. 31, 2022

(54) COMMON CONTACT SURFACES FOR USE IN THE MANUFACTURE, PACKAGING, DELIVERY, AND ASSESSMENT OF BIOPHARMACEUTICAL PRODUCTS

(71) Applicant: SIO2 MEDICAL PRODUCTS, INC., Auburn, AL (US)

(72) Inventors: Robert S. Abrams, Auburn, AL (US); Christopher Weikart, Auburn, AL (US); Ahmad Taha, Auburn, AL (US)

(21) Appl. No.: 17/425,361

(22) PCT Filed: Jan. 24, 2020
(86) PCT No.: PCT/US2020/015108

§ 371 Date: Jul. 23, 2021

Related U.S. Application Data
(60) Provisional application No. 62/797,028, filed on Jan. 25, 2019.

(51) Int. Cl. A61J 1/14; C12M 1/00

(52) U.S. Cl. CPC A61J 1/1468; C12M 23/20

(57) ABSTRACT

System of vessels, connectors, or other equipment characterized by at least some common surfaces in contact with a biopharmaceutical product during biopharmaceutical manufacturing, packaging, delivery, and assessment. Methods of producing those systems and customizing contact surfaces for use in them.

### CROSS-REFERENCE TO RELATED APPLICATION

This application claims the benefit of, priority to, and incorporates by reference in its entirety U.S. Provisional Application No. 62/797,028, filed Jan. 25, 2019.

### FIELD

The present disclosure relates to systems characterized by two or more vessels and/or connectors having a common surface in contact with a biopharmaceutical product during its manufacture, packaging, and delivery. The disclosure also relates to methods to determine and enhance the compatibility of biomaterials, biopharmaceutical compositions and their precursors and intermediates (collectively biopharmaceutical products) with a contact surface and to provide systems characterized by two or more vessels and/or connectors having a customized common surface in contact with the biopharmaceutical products or their precursors and intermediates during the manufacture, packaging and delivery of the biopharmaceutical product.

### BACKGROUND OF THE DISCLOSURE

This disclosure is broadly directed to systems of vessels or other equipment, two or more of which are characterized by common surfaces in contact with a biopharmaceutical product during its manufacture, packaging and delivery. The common surfaces reside on the walls of the vessels or other equipment in substantially all areas where those walls are in contact with the biopharmaceutical product and/or a composition comprising it, precursors or intermediates of the biopharmaceutical product, and/or biomaterials used to produce the biopharmaceutical product or for use as a therapeutic agent. Further, the disclosure relates to development of customized common surfaces that are compatible with the various biomaterials and/or biopharmaceutical products and precursors or intermediates thereof, for use on vessels or other equipment whose surfaces come in contact with those products, compositions, precursors or intermediates during the production, packaging, and delivery of a biopharmaceutical product.

A biopharmaceutical product or biologic is a medicinal product that is produced from living organisms or contains components of living organisms. Biopharmaceutical products may contain proteins that control the action of other proteins and cellular processes, genes that control production of proteins or are themselves therapeutically useful, proteins that are biologically or immunologically active in living organisms, including plants, microorganisms, viruses and animals, including humans, or cells that produce substances that suppress or activate components of the immune system or are therapeutically useful in themselves. Types of biopharmaceutical products in the disclosure include cells, proteins, (including antibodies, hormones, and enzymes), nucleic acids (including DNA, RNA and antisense oligonucleotides), vaccines, antibiotics, blood, blood components, allergens, genes, and tissues. Ultimately, biopharmaceutical products are used to treat diseases and conditions, such as cancers, rheumatoid arthritis, shingles, and Crohn's disease.

Biopharmaceutical products are made using complex manufacturing processes that typically involve genetically engineered living cells that must be frozen for storage, thawed causing minimal damage, and made to grow in a reaction vessel or bioreactor. Living cells from which biopharmaceutical products of this disclosure may be produced include, for examples, microbial cells (e.g., E. coli or yeast cells), mammalian cell lines, plant cell cultures, moss and other plants. Those cells are typically grown in bioreactors of various configurations. Once produced in those cells, the desired biopharmaceutical products must be segregated from the cultured cells that produced them and/or the media into which they are secreted, and purified all without damaging their complex, fragile structures and without microbial contamination (by e.g., bacteria, viruses, mycoplasma, cellular debris, etc).

Manufacturing a biopharmaceutical product on a large scale is typically conducted in a series of connected vessels, the precursors having upstream and downstream components. See Jozala A F et al. Brazilian J. of Microbiology 47S (2016) 51-63; Hong M S et al., Computers and Chem Engg. 110 (2018) 106-114 and Hughes B. and Hann L E, Biologics in General Medicine, (2007), Chapter 7 for an overview of the biopharmaceutical product manufacturing process. The upstream process is defined as the entire process from early cell isolation and cultivation, to cell banking and culture expansion of the cells until final harvest (termination of the culture and collection of the live cell batch, the media and the desired product). In the upstream process, cells or cell lines (cell culture) are first grown in bioreactors. Bioreactors support a range of applications including fermentation, stem cell development, strain selection and cell-line optimization, bioprocess development, early cell screening and media selection. After the desired cells and density of cells are produced in the cultures, they are cultured to produce the desired product, e.g., the biopharmaceutical product. The product may be secreted into the media by the cell, may be remain intracellular, or may be the cell itself. In each case, the product is moved to the downstream process to obtain the purified biopharmaceutical product with the desired quality or therapeutic activity.

The downstream part of a bioprocess refers to the part where the cell mass and/or media from the upstream process is processed to meet the purity and quality requirements for delivering the biopharmaceutical product in the treatment of diseases or conditions. The steps of typical downstream processing typically include: (a) Separation of biomass: separating the biomass (microbial cells) is generally carried out by centrifugation or ultra-centrifugation. If the product is the biomaterial itself, it is then recovered for processing and spent medium is discarded. If the product is extracellular, the biomass is discarded. Ultrafiltration is an alternative to the centrifugation. (b) Cell disruption: If the desired product is intracellular, the cell biomass can be disrupted so that the product is released. The solid-liquid is typically separated by centrifugation or filtration and cell debris is discarded. (c) Concentration of broth: The spent media containing the extracellular product or media containing the released product is concentrated. (d) Initial purification: According to the physico-chemical nature of the desired product, several methods for recovery of product from e.g., the clarified fermented broth or media containing the biopharmaceutical product can be used, such as precipitation or chromatography. (e) De-watering: If a low amount of product is present in a very large volume of medium, the volume is reduced by removing the media to concentrate the product. This is often done by vacuum drying or reverse osmosis. (f) Polishing: this is the final step of making the product 98 to 100% pure. The purified biological product is then typically stored in bulk prior to being formulated in a fill facility for ultimate delivery to consumers. During formulation or fill, aliquots of the bulk material are typically mixed with inert ingredients called excipients. The formulated product is then packaged in the vessels (e.g., syringes or bags) used to deliver the product to subjects in need thereof and sent to the market for the consumer use.

Each of the above steps requires vessels/connectors having surfaces that come into contact with the biopharmaceutical products (e.g., fluids comprising the biopharmaceutical product, and/or its precursors and intermediates and/or the raw biomaterials or cells used to produce, or being themselves, the biopharmaceutical product). In some of the vessels or connectors, the contact period with the biopharmaceutical product is short-lived. In others, the contact period could be longer, i.e., days or weeks, while in yet others, the contact period could be months, e.g., bulk storage and delivery devices. Moreover, the biopharmaceutical product may be in contact with various surfaces over the course of their manufacturing, packaging, and delivery. For instance, one manufacturer may supply the bioreactor or roller bottles or the single use bags in which cells producing the biopharmaceutical product are cultured, another may supply the bulk containers used to store the biopharmaceutical product; another manufacturer may supply vials or bags, or prefilled syringes for packaging of the biopharmaceutical product for delivery to subjects. Each different surface that comes into contact with a biopharmaceutical product has the potential to contaminate the material, unfavorably interact with it, or cause unintended effects on the biological activity of the biopharmaceutical product. Moreover, the material may stick to the walls of the various vessels and/or connectors causing a reduction in yield or variations in the amounts of the biomaterial product to be delivered to subjects for the treatment of diseases and conditions.

This is particularly problematic with the increased use of single-use systems comprising plastic containers. The biopharmaceutical industry in recent years is moving away from large-scale, fixed-tank facilities to flexible equipment such as single use containers in manufacture of biopharmaceutical products. Single-use systems can save space, increase flexibility in scale and space planning and, and minimize cleaning costs in development and change over. Scott Rudge, European Pharmaceutical Review, Article 50692 (2018). However, single-use containers, which are mostly made from plastics can leach out chemicals into the process solution during biopharmaceutical product manufacture. Extractables, a subset of leachables, also possible when single-use equipment undergoes heating or contact with solvents (Monika Mahajani, SDI Blog (2019). All product contact surfaces also have the potential to release extractable material into a process and affect the biomaterial or biopharmaceutical products bring produced, packaged, and delivered. Cell lines used in the manufacture of biopharmaceutical products can be particularly vulnerable to extractables and leachables (collectively, E&L). For instance, some cell lines are sensitive to a cytotoxic leachate bis(2,4-di-tert-butylphenyl)phosphate (bDtBPP) (Hammond M, et al. PDA J. Pharm. Sci. Technol. 67(2) 2013: 123-134) from the contact surface of plastic containers, while others are sensitive to the hormone-like effects of some slip agents on contact surfaces (Tappe A et al; Bioprocess International Jan. 13, 2016).

Thus, each different surface that comes into contact with a biopharmaceutical product during its manufacture, packaging, and delivery presents the potential for (a) contaminating the product (such as with E&L or elemental impurities from the plastic material), (b) causing instability of the product (such as when a protein adsorbs onto the contact surface and degrades), (c) reducing product quantity and quality, (d) reducing the shelf life of the product, (e) causing unintended effects on biological activity (such as silicon oil causing protein aggregation), and (f) causing variation of product efficacy/quality/quantity/purity of the product across batches moved across vessels with different contact surfaces.

In a biopharmaceutical manufacturing, purchasing and delivery process, regulatory bodies require a risk assessment to be made for E&L from every contact surface, including factors such as nature of the extractable species, process fluid, contact time, and contact temperature (see Smithers Rapra E&L Conference News; Introduction to Extractables and Leachables Testing (2015). Preparing such E&L assessments for each different surface can be a time-consuming process. These times are exacerbated for vessels where shelf life and long-term storage are important.

The FDA and other regulatory agencies worldwide also have provided guidelines for good manufacturing practices (GMP) for the production of biopharmaceutical products under an appropriate system for managing quality. See e.g., Q7 Good Manufacturing Practice Guidelines for Active Pharmaceutical Ingredients (APIs); FDA Document 71518 (2016); and Document 112426 (2018). Adhering to the regulatory guidelines requires careful validation of every process and equipment to be used in the manufacture, packaging, and delivery of a biopharmaceutical product. Thus, using a range of vessels having different contact surfaces, during the manufacture, packaging, and delivery process of a biopharmaceutical product, can greatly slow down the process of obtaining regulatory approvals for the production, packaging and delivery of that biopharmaceutical product.

Further, any amendments to the manufacturing process, or deviations from established protocols, including changes in equipment or materials, which may affect product quality and/or reproducibility of the process must be validated to ensure that there are no detrimental effects on the material's fitness for use. In this regard, for example, the GMP guidelines provide for “Change Control”, wherein, any changes to the manufacturing process require regulatory filings and prior regulatory approval. See e.g., Quality Systems Approach to Pharmaceutical CGMP Regulations; FDA Document 71023 (2006) and Pharma Change Control; The Executive Briefing Series; FDA News (2013). Changes in manufacturing processes and equipment can also lead to delays in the time to manufacture if equipment with different materials have to be evaluated and validated separately in order to comply with guidelines.

Preferred biopharmaceutical products of this disclosure are proteins, such as therapeutic proteins and antibodies, and the like. The amino acid interactions that cause such proteins and antibodies to fold and induce therapeutic effects in the body also cause unwanted adsorption of proteins with surfaces and interfaces with which they may come into contact. Protein adsorption can lead to irreversible denaturing and aggregation. Other preferred biopharmaceutical products of the disclosure are biomaterials, such as cells useful in cell-based therapies. These biomaterial may also be affected by the surface with which they come into contact. Yet other biopharmaceutical products of the disclosure may be nucleic acids, including DNA, RNA, and antisense oligonucleotides, which may also be affected by the surface with which they come into contact.

### SUMMARY OF THE DISCLOSURE

There is accordingly a need to minimize the different surfaces with which a biopharmaceutical product, its intermediates and precursors comes into contact in order to reduce potential issues with product efficacy, quality, purity, shelf life, etc. There is also a need to optimize and harmonize upstream and downstream processes involved in the manufacturing, packaging and delivery of biopharmaceutical products, in order to produce a high yield, high quality, highly pure product in a timely and cost-effective manner. A direct advantage of biopharmaceutical product manufacturing, packaging and delivery systems with common contact surfaces is the reduced time required for validation of these systems by regulatory agencies, and thus a faster and more efficient path to market for the ultimate product. Further, there is a need to customize the surfaces that come into contact with a particular biopharmaceutical product, its intermediates and precursors, in order to increase its compatibility with that product and reduce or avoid denaturation or adsorption or both as well as other effects that result from surface biopharmaceutical product contact.

In one aspect, a system of the present disclosure comprises at least two vessels or connectors used in the manufacture, packaging or delivery of a biopharmaceutical product, each of said vessels comprising walls defining inner cavities, the vessels being optionally open or capable of being opened on one end or on one side; the walls of each of said vessels having inner surfaces facing the cavities, the inner surfaces being characterized by a common surface residing thereon in substantially all areas where the inner surfaces are in contact with the biopharmaceutical product, the common surface comprising a tie coating or layer, a barrier coating or layer, and optionally, a pH protective coating or layer;
the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
the barrier coating or layer comprising SiOx, wherein x is from 1.5 to 2.9, the barrier coating or layer being from 2 to 1000 nm thick, the barrier coating or layer having an outer surface facing the inner surface of the tie coating or layer and having an inner surface facing the cavities; and
the pH protective coating or layer comprising SiOxCy or SiNxCy wherein x is from about 0.5 to about 2.4 and y is from about 0.6 to about 3, the pH protective coating or layer having an outer surface facing the inner surface of the barrier coating or layer and having an inner surface facing the cavities.

In preferred aspects of this disclosure, the at least two vessels comprise a first vessel useful for holding a bulk amount of the biopharmaceutical product and a second vessel useful for administering aliquots of that bulk amount to a subject in need thereof.

In other preferred aspects, the at least two vessels comprise vessels useful in the culturing of cells to produce the biopharmaceutical product.

In other aspects of this disclosure, the at least two vessels/connectors comprise the majority or, preferably all, of the vessels/connectors useful in the culturing of cells to produce the biopharmaceutical product. In other aspects, the at least two vessels/connectors comprise the majority or, preferably all, of the vessels/connectors useful in the culturing of the cells to produce the biopharmaceutical product and the majority or, preferably all, of the vessels/connectors useful in the bulk storage of those products and their packaging for delivery to subjects in need thereof.

A system of the present disclosure has several advantages. These include, for example, a common contact surface of at least two if the vessels and/or connectors, which can reduce potential issues with the yield, quality, purity and efficacy of the biopharmaceutical product. A common surface on at least two of the vessels and connectors, and preferably more of them, can reduce the time required to validate each contact surface and having it approved by a regulatory agency (such as FDA). This in turn would lead to a faster path to market approval for the biopharmaceutical product. Thus, a system of the present disclosure provides a novel solution to increasing the efficacy of biopharmaceutical product manufacturing, packaging, and delivery processes, while simultaneously ensuring the production of a high yield, high quality biopharmaceutical product.

Particular embodiments of the disclosure are set forth in the following numbered paragraphs:

1. A system comprising at least two vessels or connectors used in the manufacture, packaging, or delivery of a biopharmaceutical product; wherein each of said vessels comprise walls defining inner cavities, the vessels being optionally open or capable of being opened on one end or on one side; the walls of each of said vessels having inner surfaces facing the cavities, the inner surfaces being characterized by a common surface residing thereon in substantially all areas where the inner surfaces are in contact with the biopharmaceutical product, the common surface comprising a tie coating or layer, a barrier coating or layer, and optionally, a pH protective coating or layer;
the tie coating or layer comprising SiOₓC_{y} or SiNₓC_{y} wherein x is from about 0.5 to about 2.4 and y is from about 0.6 to about 3, the tie coating or layer having an outer surface facing the walls of the vessels and having an inner surface facing the cavities;
the barrier coating or layer comprising SiOₓ, wherein x is from 1.5 to 2.9, the barrier coating or layer being from 2 to 1000 nm thick, the barrier coating or layer having an outer surface facing the inner surface of the tie coating or layer and having an inner surface facing the cavities; and
the pH protective coating or layer comprising SiOₓC_{y} or SiNₓC_{y} wherein x is from about 0.5 to about 2.4 and y is from about 0.6 to about 3, the pH protective coating or layer having an outer surface facing the inner surface of the barrier coating or layer and having an inner surface facing the cavities.
   2. The system of paragraph 1, wherein the vessel is selected from a group consisting of one or more of the following: 384 well plates; 96 well plates; bioreactors; blood sample collection tubes; bottles; bulk storage vessels; cannulas; capture columns; cartridges; catheters; cell bank vials; cell separators; cell vials; centrifugal pumps; centrifuge tubes; chromatography columns; chromatography vials; clarifiers; closures; container closure systems; cryopreservation vessels; culture bottles; delivery containers; diafiltration equipment; dispensing units, ELISA plates; elution bags; evacuated blood sample collection tubes; fill/finish equipment; filtration equipment; freeze dryer equipment; harvest vessels; shakers; in-process analysis instruments; intermediate columns; intravenous (IV) bags; media bags; media bottles; media vessels; membrane chromatography columns; microplates; microtiter plates; microwell plates; mixing bags; monitoring devices; multiple-use bioreactors; package filling apparatus; pumps; petri dishes; pipette tips; plates; plungers; polish columns; pre-filled syringes; prefilled syringe with luer lock fittings; primary packaging; production bioreactors; production fermenters; roller bottles; sample collection tubes; sampling vessels; seed fermenters; separators; shake flasks; single-use bioreactors; slides; spinner flasks; staked needle pre-filled syringes; storage bags; sterilizers; stock culture vials; storage containers; syringes (prefilled); tanks; terminal reactors; transfer bags; ultrafiltration/diafiltration equipment; ultrafiltration equipment; upstream bioreactors; valves; vials; viral filtration equipment; viral inactivation vessels; and wave bioreactors.
   3. The system of paragraph 2, wherein at least one of the vessels is selected from a group consisting of bioreactors, pumps, cell bank vials, harvest vessels, stock culture vials, fermenters, shake flasks, shakers, columns, separators, bags, beakers, tanks, cylinders, bulk storage units or containers, single use bags, roller bottles, storage tubes, and sampling vessels.
   4. The system of paragraph 2, wherein at least one of the vessels is selected from a group consisting of syringes, dispensing units, vials and intravenous (IV) bags.
   5. The system of any one of paragraphs 1-4, wherein the at least two vessels comprise (a) a first vessel useful for holding a bulk amount of the biopharmaceutical product; and (b) a second vessel useful for administering a composition comprising at least a portion of said biopharmaceutical product to a subject in need thereof.
   6. The system of paragraph 5, further comprising one or more connectors connecting one or more of the vessels to one or more of another vessel or connector, wherein at least one of said vessels in the one or more series has a wall whose inner surface is characterized by a surface residing thereon in substantially all the areas that come into contact with the biopharmaceutical product during its production, the surface being common to the innermost surface of the first and second vessels.
   7. The system of paragraph 6, wherein one of the vessels in the one or more series of vessels is independently selected from a group consisting of bioreactors, fermenters, pumps, roller bottles, flasks, shakers, separators, bags, beakers, sampling vessels, cylinders, pipette tips, slides, and vials.
   8. The system of paragraph 6 or 7, wherein at least one of the vessels in the one or more series of vessels is connected through one or more connectors, to another of the vessels in the one or more series of vessels, wherein at least one of said connectors comprises walls defining an inner cavity, the walls having an inner surface characterized by a surface residing thereon in substantially all areas that come into contact with the biopharmaceutical product during its production, the surface being common to the innermost surface of the first and second vessels.
   9. The system of paragraph 8, wherein each connector is independently selected from a group consisting of tubes, tubings, valves, and pipes.
   10. The system of paragraph 5, further comprising one or more series of vessels useful in the production of said bulk amount of the biopharmaceutical product, wherein at least one of said vessels in the one or more series has a wall whose inner surface is characterized by a surface residing thereon in substantially all the areas that come into contact with the biopharmaceutical product during its production, the surface being not common to that of the first and second vessels, wherein the surface residing on said wall in substantially all the areas that come into contact with the biopharmaceutical product during its production, is common to the surface on the wall of at least one of another of said vessels in the one or more series.
   11. The system of paragraph 10, wherein the common surface comprises a tie coating or layer, a barrier coating or layer, and optionally, a pH protective coating or layer;
the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
the barrier coating or layer comprising SiOx, wherein x is from 1.5 to 2.9, the barrier coating or layer being from 2 to 1000 nm thick, the barrier coating or layer having an outer surface facing the inner surface of the tie coating or layer and having an inner surface facing the cavities; and
the pH protective coating or layer comprising SiOxCy or SiNxCy wherein x is from about 0.5 to about 2.4 and y is from about 0.6 to about 3, the pH protective coating or layer having an outer surface facing the inner surface of the barrier coating or layer and having an inner surface facing the cavities.
   12. A system comprising at least two vessels or connectors useful in assessing one or more of the following characteristics: quality, purity, and integrity of one or more of a biopharmaceutical product, wherein each of said vessels comprises walls defining inner cavities, the vessels being optionally open or capable of being opened on one end or on one side; the walls of each of said vessels having inner surfaces facing the cavities, the inner surfaces being characterized by common surfaces residing thereon in substantially all areas where the inner surfaces are in contact with the biopharmaceutical product, comprising a tie coating or layer, a barrier coating or layer, and optionally, a pH protective coating or layer;
the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
the barrier coating or layer comprising SiOx, wherein x is from 1.5 to 2.9, the barrier coating or layer being from 2 to 1000 nm thick, the barrier coating or layer having an outer surface facing the inner surfaces of the tie coating or layer and having an inner surface facing the cavities; and
the pH protective coating or layer comprising SiOxCy or SiNxCy wherein x is from about 0.5 to about 2.4 and y is from about 0.6 to about 3, the pH protective coating or layer having an outer surface facing the inner surfaces of the barrier coating or layer and having an inner surface facing the cavities.
   13. The system of paragraph 12, wherein the common surface is not identical to the common surface of the first and second vessels of paragraph 5, the common surface of the one or more vessels of paragraph 9, or either.
   14. The system of paragraph 12, wherein the common surface is identical to one or both of the common surface of the first and second vessels of paragraph 5, and the common surface of the one or more vessels of paragraph 9.
   15. The system of paragraph 11 or 12, wherein each vessel is selected from a group consisting of microplates, microtiter plates, microwell plates, and petri dishes.
   16. The system of any one of paragraphs 1-15, wherein the biopharmaceutical product is selected from the group consisting of one of more of the following: an antibody, a bulk biopharmaceutical formulation, a cell culture formulation, a cell suspension, a culture fluid, a biopharmaceutical product, a biopharmaceutical substance, a biopharmaceutical formulation, an expression vector/host formulation, a harvested cell formulation, a host cell composition, a host cell contaminant, a mobile phase, a monoclonal antibody formulation, a product stream, a seed train composition, a stationary phase, a peptide or protein formulation, and a nucleic acid formulation.
   17. A method for producing a system comprising at least two vessels or connectors useful in the manufacture, packaging, or delivery of a biopharmaceutical product, each of said vessels or connectors comprising walls defining inner cavities, the vessels or connectors being optionally open or capable of being opened on one end or on one side; the walls of each of said vessels or connectors having inner surfaces facing the cavities, the method comprising:
providing a biopharmaceutical product;
providing a provisional contact surface;
determining if the biopharmaceutical product is compatible with the provisional contact surface;
modifying the provisional contact surface to improve its compatibility with the biopharmaceutical product, thereby producing a customized contact surface; and
   using the customized contact surface to produce the system such that the inner surfaces of the vessels/connectors are characterized by the customized common surface residing thereon in substantially all areas where the inner surfaces are in contact with the biopharmaceutical product, the customized common surface comprising a tie coating or layer, a barrier coating or layer, and optionally, a pH protective coating or layer;
the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
the barrier coating or layer comprising SiOx, wherein x is from 1.5 to 2.9, the barrier coating or layer being from 2 to 1000 nm thick, the barrier coating or layer having an outer surface facing the inner surface of the tie coating or layer and having an inner surface facing the cavities; and
the pH protective coating or layer comprising SiOxCy or SiNxCy wherein x is from about 0.5 to about 2.4 and y is from about 0.6 to about 3, the pH protective coating or layer having an outer surface facing the inner surface of the barrier coating or layer and having an inner surface facing the cavities.
   18. The method of paragraph 17, wherein the biopharmaceutical product is selected from the group consisting of one of more of the following: an antibody, a bulk biopharmaceutical formulation, a cell culture formulation, a cell suspension, a culture fluid, a biopharmaceutical product, a biopharmaceutical substance, a biopharmaceutical formulation, an expression vector/host formulation, a harvested cell formulation, a host cell composition, a host cell contaminant, a mobile phase, a monoclonal antibody formulation, a product stream, a seed train composition, a stationary phase, a peptide or protein formulation, and a nucleic acid formulation.
   19. The method of paragraph 17 or 18, where in the provisional contact surface has a water contact angle of less than 90°.
   20. The method of any one of paragraphs 17-19, wherein provisional contact surface has less than 1% H bond donor groups as determined by x-ray photoelectron spectroscopy (XPS) and optionally hydrogen forward scattering (HFS) analysis or Rutherford backscattering (RBS) analysis.
   21. The method of any one of paragraphs 17-20, wherein the provisional contact surface has at least 1% H bond acceptor groups as determined by XPS and optionally HFS or RBS analysis.
   22. The method of any one of paragraphs 17-21, wherein the provisional contact surface has less than 1% anion and cation groups as determined by XPS.
   23. The method of any one of paragraphs 17-22, wherein the provisional contact surface is essentially free of metal or metalloid atoms other than silicon as determined by XPS.
   24. The method of any one of paragraphs 17-23, wherein the provisional contact surface is a plasma-enhanced chemical vapor deposition (PECVD) coating.
   25. The method of any one of paragraphs 17-24, wherein the provisional contact surface has the following statistical ratios of silicon, oxygen, carbon, and hydrogen atoms, determined by XPS and optionally HFS or RBS: Si=1:O=x:C=y:H=z, in which x is from about 1.5 to about 2.9, y is from about 0 to about 1, and z is within the range from about 0 to about 4.
   26. The method of any one of paragraphs 17-24, wherein the provisional contact surface has the following statistical ratios of silicon, oxygen, carbon, and hydrogen atoms, determined by XPS and optionally HFS or RBS: Si=1:O=x:C=y:H=z, in which x is from 0.5 to 2.4, y is from 0.6 to 3, and z is from 2 to 9.
   27. The method of any one of paragraphs 17-24, wherein the provisional contact surface has the following statistical ratios of silicon, oxygen, carbon, and hydrogen atoms, determined by XPS and optionally HFS or RBS: Si=1:O=x:C=y:H=z, in which x is from 1.5 to 2.9, y is about 0, and z is about 0.
   28. The method of any one of paragraphs 17-27, wherein the vessel is selected from a group consisting of one or more of the following: 384 well plates; 96 well plates; bioreactors; blood sample collection tubes; bottles; bulk storage vessels; cannulas; capture columns; cartridges; catheters; cell bank vials; cell separators; cell vials; centrifugal pumps; centrifuge tubes; chromatography columns; chromatography vials; clarifiers; closures; container closure systems; cryopreservation vessels; culture bottles; delivery containers; diafiltration equipment; dispensing units, ELISA plates; elution bags; evacuated blood sample collection tubes; fill/finish equipment; filtration equipment; freeze dryer equipment; harvest vessels; shakers; in-process analysis instruments; intermediate columns; intravenous (IV) bags; media bags; media bottles; media vessels; membrane chromatography columns; microplates; microtiter plates; microwell plates; mixing bags; monitoring devices; multiple-use bioreactors; package filling apparatus; pumps; petri dishes; pipette tips; plates; plungers; polish columns; pre-filled syringes; prefilled syringe with luer lock fittings; primary packaging; production bioreactors; production fermenters; roller bottles; sample collection tubes; sampling vessels; seed fermenters; separators; shake flasks; single-use bioreactors; slides; spinner flasks; staked needle pre-filled syringes; storage bags; sterilizers; stock culture vials; storage containers; syringes (prefilled); tanks; terminal reactors; transfer bags; ultrafiltration/diafiltration equipment; ultrafiltration equipment; upstream bioreactors; valves; vials; viral filtration equipment; viral inactivation vessels; and wave bioreactors.
   29. The method of any one of paragraphs 17-28, wherein the method of modifying the provisional contact surface of a vessel to increase its compatibility with a particular biopharmaceutical product to produce a customized contact surface comprises at least one of the following steps:
reducing the water contact angle of the provisional contact surface by about 10%-90% of the original water contact angle;
reducing the percentage of H bond donor groups of the provisional contact surface by about 10%-90%, as determined by XPS analysis and optionally HFS analysis or RBS analysis;
increasing the percentage of H bond acceptor groups of the provisional contact surface by about 10%-90%, as determined by XPS analysis and optionally HFS analysis or RBS analysis;
reducing the percentage of anion groups, cation groups, or both, of the provisional contact surface by about 10%-90%, as determined by XPS analysis;
reducing the percentage of metal or metalloid atoms, other than silicon, of the provisional contact surface by about 10%-90%, as determined by XPS.

Further aspects of the disclosure will be apparent from the description and claims of this specification.

### BRIEF DESCRIPTION OF THE DRAWINGS

FIG. 1a compares the amount of IgG protein adsorbed (ng/cm²) on the contact surface of coated polymer tubes (bilayer and trilayer of this disclosure) and an uncoated polymer cyclic olefin polymer (COP) container. FIG. 1b compares the amount of IgG protein retained (ng/cm²) on the contact surface of coated polymer tubes (bilayer and trilayer of this disclosure) and an uncoated polymer cyclic olefin polymer (COP) container.

### DEFINITIONS

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description above.

Exemplary methods and materials are described herein, although methods and materials similar or equivalent to those described herein can also be used in the practice or testing of the various aspects and embodiments. The materials, methods, and examples are illustrative only and not intended to be limiting.

The word “comprising” according to any embodiment does not exclude other elements or steps. The indefinite article “a” or “an” does not exclude a plurality unless indicated otherwise. Singular form includes the plural.

Whenever a parameter range is indicated, it is intended to disclose the parameter values given as limits of the range and all values of the parameter falling within said range.

Reference to “about” a value or parameter herein includes (and describes) embodiments that are directed to that value or parameter per se. For example, description referring to “about X” includes description of “X.” As used herein, the term “about” permits a variation of ±10% within the range of the significant digit.

Where aspects or embodiments are described in terms of a Markush group or other grouping of alternatives, the present disclosure encompasses not only the entire group listed as a whole, but each member of the group individually and all possible subgroups of the main group, and also the main group absent one or more of the group members. The present disclosure also envisages the explicit exclusion of one or more of any of the group members or the inclusion of other members, not listed in the Markush group.

As used herein, the term “system” refers to a set of vessels/connectors or several sets of vessels/connectors working together in the manufacture, packaging, and/or delivery of a biopharmaceutical product. A system may also comprise a set of vessels/connectors useful for testing the quality of the manufacturing process and/or biopharmaceutical product produced using that process, including testing the quality of the biopharmaceutical product during and after packaging and storage. A system comprises vessels/connectors, each of which has surfaces that contact the biopharmaceutical product and/or its intermediates or precursors. In some embodiments, the system comprises two or more of the vessels/connectors used in the upstream process in the biopharmaceutical product manufacturing process. In some embodiments, the system comprises two or more of the vessels/connectors useful in the downstream process in the biopharmaceutical product manufacturing process. In some embodiments, the system comprises two or more of the vessels/connectors required in the quality control of the biopharmaceutical product manufacturing, packaging and delivery process. In yet other embodiments, the system comprises two or more of the vessels/connectors useful in some combination of the upstream and downstream processes in the biopharmaceutical product manufacturing, packaging and delivery process, as well as the vessels/connectors used in the quality control of the process and its various stages.

As used herein, the term “vessel” can be any type of article for use in the manufacturing, packaging, delivery, and/or quality control of the biopharmaceutical product. A vessel comprises walls defining inner cavities. A vessel in the context of the present disclosure, is optionally open or capable of being opened on one end or on one side. The walls of each vessel have inner surfaces facing the cavities, the inner surfaces being characterized by a surface residing thereon in substantially all areas where the inner surfaces are in contact with the biopharmaceutical product. A vessel/connector in the context of the present disclosure can have one or more openings, for example one opening or two openings. A vessel may have rigid walls, or flexible walls, or a combination of both. A rigid vessel may be made of stainless steel and the like, such as a stainless steel bioreactor or glass or metal roller bottle. A flexible vessel may be a single use disposable bag, such as a plastic bioreactor or storage bag, alone or used with a vessel having rigid or flexible walls.

In some embodiments, the series of vessels in the upstream process include any vessel that comes in contact with the biopharmaceutical product or its precursors; and any fluid solvent, culture media, etc.

Some non-limiting examples of a vessel of the present disclosure are: a 384 well plate; 96 well plate; bioreactor; blood sample collection tube; bottle; bulk storage vessel; cannula; capture column; cartridge; catheter; cell bank vial; cell separator; cell cial; centrifugal pump; centrifuge tube; Chromatography Column; chromatography vial; clarifier; closure; container closure system; cryopreservation vessel; culture bottle; delivery container; diafiltration equipment; ELISA plate; elution bag; evacuated blood sample collection tube; fill/finish equipment; filtration equipment; freeze dryer equipment; harvest vessel; shaker; in-process analysis instruments; intermediate column; intravenous (IV) bag; media bag; media bottle; media vessel; membrane chromatography column; microplate; microtiter plate; microwell plate; mixing bag; monitoring device; multiple-use bioreactor; package filling apparatus; pump; petri dish; pipette tip; plate; plunger; polish column; pre-filled syringe; prefilled syringe with luer lock fitting; primary packaging; production bioreactor; production fermenter; roller bottle; sample collection tube; sampling vessel; seed fermenter; separator; shake flask; single-use bioreactor; slide; spinner flask; staked needle pre-filled syringe; storage bag; sterilizer; stock culture vial; storage container; syringe (prefilled); tank; terminal reactor; transfer bag; ultrafiltration/diafiltration equipment; ultrafiltration equipment; upstream bioreactor; valve; vial; viral filtration equipment; viral inactivation vessel; or wave bioreactor.

As used herein, the term “biopharmaceutical”, “biopharmaceutical product” and the like refer to a therapeutic or prophylactic drug or product produced from living organisms (biomaterials) or containing the components of living organisms, and/or products produced by means that involve extraction from a native (non-engineered) biological source and their precursors and intermediates, including proteins (including antibodies, hormones, and enzymes), nucleic acids (including DNA, RNA or antisense oligonucleotides), vaccines, antibiotics, blood, blood components, cells, allergens, genes, and tissues. Such products may be used for therapeutic, prophylactic, or diagnostic purposes. As used herein, the term “biomaterial” (as a part of biopharmaceutical products) refers to the raw material (including solvents, culture media, mobile phase, stationary phase, and the like) used to produce the biopharmaceutical product, cells, cell lines, cell suspension, inoculum, seed train, product stream, cell culture, expression vector/host formulation, harvested cells, host cell composition, host cell contaminant, biomass, and the like, and the biomaterial itself when used, for example, in cell-based therapies.

As used herein, the term “surface” refers to inner surfaces of vessels/connectors which come into contact with the biopharmaceutical product. In particular, a vessel/connector of the present disclosure comprises walls defining inner cavities, the vessels/connectors being optionally open or capable of being opened on one end or on one side; the walls of each of said vessels/connectors having inner surfaces facing the cavities, the inner surfaces being characterized by surfaces residing thereon in substantially all areas where the inner surfaces are in contact with the biopharmaceutical product.

As used herein, the term “common surface” refers to an inner surface of a vessel/connector that is identical to the inner surface of at least one other vessel/connector used in the manufacturing, packaging, and delivery of a biopharmaceutical product.

The terms pertaining to the various coatings or layers, i.e., the tie coating or layer, barrier coating or layer, and pH coating or layer, and methods of making and applying the coating or layer, e.g. by plasma enhanced chemical vapor deposition (PECVD) are described in U.S. Publ. Appl. 2018-0334545 A1; U.S. Pat. Nos. 10,016,338, 9,937,099, and 9,554,968 e.g., directed to a trilayer PECVD coating; U.S. Pat. Nos. 9,878,101, 9,863,042, 9,764,093, 9,458,536, 9,272,095 (hydrophobic), and U.S. Pat. No. 7,985,188 also directed to PECVD coatings; U.S. Pat. No. 9,662,450 e.g., directed to plasma treatment of polymer surfaces; U.S. Pat. No. 9,545,360 directed to a saccharide protective coating for a pharmaceutical package; U.S. Publ. Appl. 2015-0297800 A1 e.g., directed to a pharmaceutical package, PCT Application No. PCT/US2020/012638 directed to flexible bags, and U.S. Provisional Application No. 62/929,668 e.g., directed to blood collection tubes. Each of these documents is incorporated herein by reference as a whole into this specification.

As used herein, the term “connector” refers to equipment which provides a reliable connection and fluid transfer, typically sterile, between two separate process components or vessels in biopharmaceutical product manufacturing operations. A connector may be gendered or genderless, and may be suitable for single use or multiple use. A connector may be used to connect two rigid vessels to each other, two flexible vessels to each other, or a rigid vessel to a flexible vessel. A connector may be a tube, tubing, valve, pipe, etc. See e.g., Proctor G, Parker Bioprocess Filtration Team report (Oct. 15, 2019) for various types of connectors.

Metalloids other than silicon as used in the present disclosure are boron, germanium (Ge), arsenic (As), antimony (Sb), tellurium (Te), and Astatine (At). Metals as used in this specification are any of the commonly recognized metallic elements, including but not limited to alkali metals, alkaline earth metals, transition metals, lanthanoids and actinoids.

As used herein, the term “upstream process” in a biopharmaceutical manufacturing, packaging, and delivery process refers to the typical bioprocessing phase in which cell lines are generated and biomass is produced at appreciable scale. Upstream processing is the growth of either bacterial or cell culture-based products, referred to as microbial fermentation or mammalian cell culture, respectively. In the upstream process, the inoculum is developed (by isolating and purifying cells), growth medium is developed, and the growth kinetics are improved for mass-scale product generation. Once the appropriate cell density is reached, the culture is moved to the downstream process. For commercial production, the upstream process may take place in one or more vessels (such as bioreactors) in a volume ranging from a 1,000 L to 10,000 L. The starter culture is typically grown in a series of passages before inoculating these large production vessels.

In the upstream process, the contact surface in vessel may come into contact with the biopharmaceutical product, including the intermediates or precursors thereof, and/or the biomaterial used to generate the biopharmaceutical products or itself the biopharmaceutical product. For example, the precursor of a protein biopharmaceutical product may be an unfolded protein, a polypeptide, an unprocessed protein without or with partial post-processing chemical modifications, intermediate forms of the protein, etc. The biomaterial may be cells, inoculum of cells, cell cultures, cell lines, biomass, and the like. Examples of vessels in the one or more series of vessels used in the upstream process include bioreactors, fermenters, pumps, roller bottles, flasks, shakers, columns, separators, bags, beakers, sampling vessels, cylinders, pipette tips, slides, vials, etc.

As used herein, the term “downstream process” in a biopharmaceutical product manufacturing, packaging, and delivery process typically comprises the following non-limiting steps: (a) Removal of insoluble: the product is captured as a solute in a particulate-free liquid, for example the separation of cells, cell debris or other particulate matter from fermentation broth containing an antibiotic. Typical operations to achieve this are filtration, centrifugation, sedimentation, precipitation, flocculation, electro-precipitation, and gravity settling. Additional operations may be used, such as grinding, homogenization, or leaching, in order to recover products from solid sources such as plant and animal tissues. (b) Product isolation is the removal of those components whose properties vary considerably from that of the desired product. For most products, water is the chief impurity and isolation steps are designed to remove most of it, reducing the volume of material to be handled and concentrating the product. Product isolation operations include the steps of solvent extraction, adsorption, ultrafiltration, and precipitation are some of the unit operations. (c) Product purification is done to separate those contaminants that resemble the product in physical and chemical properties. Consequently, steps in this stage are expensive to carry out and require sensitive and sophisticated equipment. This stage contributes a significant fraction of the entire downstream processing expenditure. Examples of purification operations include affinity, size exclusion, reversed phase chromatography, ion-exchange chromatography, crystallization and fractional precipitation. (d) Product polishing describes the final downstream processing step which end with packaging of the product in a form that is stable, easily transportable and convenient. Typical operations include crystallization, desiccation, lyophilization and spray drying. Depending on the product and its intended use, polishing may also include operations to sterilize the product and remove or deactivate trace contaminants and viruses which might compromise product safety. Such operations might include the removal of viruses or depyrogenation.

All references cited are incorporated in their entireties for any purpose (this specification controls where there are inconsistencies).

### DETAILED DESCRIPTION

The present disclosure in some embodiments will now be described more fully. This disclosure can, however, be embodied in many different forms and should not be construed as limited to the embodiments set forth here. Rather, these embodiments are examples. Each embodiment described herein may be used individually or in combination with any other embodiment described herein.

### Downstream Biopharmaceutical Product Manufacturing, Packaging and Delivery Systems

One aspect of the disclosure is a system with two or more vessels comprising common contact surfaces that may be used in downstream processes for manufacturing, packaging, and delivering a biopharmaceutical product.

In some embodiments of this aspect of the disclosure, a system of the disclosure comprises at least a first vessel for holding a bulk amount of a biopharmaceutical product and a second vessel useful for administering a composition comprising at least a portion of said biopharmaceutical product to a subject in need thereof. Examples of the first vessel are tanks, cylinders, bulk storage units or containers, single use bags, roller bottles, storage tubes, etc. Examples of the second vessel are syringes, dispensing units, bags, vials, intravenous (IV) bags, etc.

Each of the vessels in this illustrative embodiment comprises walls defining inner cavities, the vessels being optionally open or capable of being opened on one end or on one side; the walls of each of said vessels having inner surfaces facing the cavities, the inner surfaces being characterized by a common surface residing thereon in substantially all areas where the inner surfaces are in contact with the biopharmaceutical product, the common surface comprising a tie coating or layer, a barrier coating or layer, and optionally, a pH protective coating or layer;
the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
the barrier coating or layer comprising SiOx, wherein x is from 1.5 to 2.9, the barrier coating or layer being from 2 to 1000 nm thick, the barrier coating or layer having an outer surface facing the inner surface of the tie coating or layer and having an inner surface facing the cavities; and
the pH protective coating or layer comprising SiOxCy or SiNxCy wherein x is from about 0.5 to about 2.4 and y is from about 0.6 to about 3, the pH protective coating or layer having an outer surface facing the inner surface of the barrier coating or layer and having an inner surface facing the cavities.

In some embodiments, the contact surfaces of some vessels and equipment in the downstream process will not be characterized by a contact surface in common with that of the other vessels in the downstream assembly. For example, filters, purification combs, chromatography columns, and the like which possess chemically modified ligands on their surface in order to capture impurities and/or the biopharmaceutical materials, may not have the same contact surface as the contact surface present in other vessels of the downstream process, such as, tanks, cylinders, bulk storage units or containers, single use bags, roller bottles, storage tubes, syringes, dispensing units, vials, and the like. Similarly, other vessels in the downstream process may not have surfaces in common. However, in preferred embodiments, multiple and in some cases the majority of the vessels and connectors used the downstream process will share a common surface.

### Upstream Biopharmaceutical Manufacturing Systems

An aspect of the disclosure is a system with two or more vessels comprising common contact surfaces that may be used in upstream processes for manufacturing a biopharmaceutical product.

In some embodiments in this aspect of the disclosure, a system of the disclosure comprises one or more series of vessels useful in the production of said biopharmaceutical product, wherein at least one of said vessels in the one or more series has an inner wall characterized by a surface residing thereon in substantially all the areas that come into contact with the biopharmaceutical product during its production, the surface being common to the innermost surface of another of the vessels and connectors used in the upstream processes or downstream processes. For example, in some embodiments, the upstream process may comprise one or more series of vessels useful in the production of the biopharmaceutical product, wherein at least one of said vessels in the one or more series has a wall whose inner surface is characterized by a surface residing thereon in substantially all the areas that come into contact with the biopharmaceutical product during its production, the surface being not common to that of any of the vessels or connectors in the downstream process but, wherein the surface residing on said wall in substantially all the areas that come into contact with the biopharmaceutical product during its production, is common to the surface on the wall of at least one of another of said vessels in the one or more series of vessels or connectors used in the upstream process. For example, each vessel in a series of vessels in the upstream process, that may be used for growing or harvesting cells (i.e., the production containers or bioreactors) that produce the desired pharmaceutical may have an identical common contact surface. However, this surface can differ from the contact surface present in the vessels of the downstream process that hold or deliver the biopharmaceutical product to subjects in need thereof.

In other embodiments, the common surface is shared by one or more vessels or connectors used in the upstream and downstream processes. In one preferred embodiment, the common surface is shared by one or more of the vessels used to culture the cells that produce the biopharmaceutical product (e.g., one or more, and preferably all of the bioreactors) and the vessels to store the bulk biopharmaceutical product and preferably also the vessels used to deliver the biopharmaceutical product to subjects in need thereof. More preferably, the common surface is also shared by the various connectors and other equipment that link the bioreactors together and with the bulk containers and the delivery vessels.

In the above aspects, the common surface in some embodiments comprises a tie coating or layer, a barrier coating or layer, and optionally, a pH protective coating or layer; the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities; the barrier coating or layer comprising SiOx, wherein x is from 1.5 to 2.9, the barrier coating or layer being from 2 to 1000 nm thick, the barrier coating or layer having an outer surface facing the inner surface of the tie coating or layer and having an inner surface facing the cavities; and the pH protective coating or layer comprising SiOxCy or SiNxCy wherein x is from about 0.5 to about 2.4 and y is from about 0.6 to about 3, the pH protective coating or layer having an outer surface facing the inner surface of the barrier coating or layer and having an inner surface facing the cavities.

### Connectors

In some embodiments, one or more vessels in the upstream or downstream processes may be connected to each other. In yet other embodiments, a connector may connect one or more vessels in the upstream process to one or more vessels in the downstream process, such as in a continuous manufacturing, packaging, and/or delivery system. In all embodiments, examples of connectors include tubes, tubings, valves, pipes, and the like.

In some embodiments, a connector provides a sterile connection between two processing units. In some embodiments, a connector provides a sterile connection between more than two processing units. For example, a series of vessels may be connected to each other via connectors, wherein the series may have multiple vessels. In some embodiments, the connectors may connect an array of vessels serially to each other. In some embodiments, the one or more connectors comprise walls defining an inner cavity, the walls having an inner surface characterized by a surface residing thereon in substantially all areas that come into contact with the biopharmaceutical, the product, intermediates, or processes or biomaterials during production, the surface being common to the innermost surface of another upstream or downstream process, vessel or connector.

### Quality Control Equipment

An aspect of the disclosure is a system comprising one or more series of vessels useful in assessing one or more of the following characteristics: quality, purity, and integrity of one or more of (i) a composition comprising the biopharmaceutical product; (ii) a precursor of the biopharmaceutical product; and (iii) a biomaterial used to produce the biopharmaceutical product; wherein each of said vessels comprises walls defining inner cavities, the vessels being optionally open or capable of being opened on one end or on one side; the walls of each of said vessels having inner surfaces facing the cavities, the inner surfaces being characterized by common surfaces residing thereon in substantially all areas where the inner surfaces are in contact with the biopharmaceutical, comprising a tie coating or layer, a barrier coating or layer, and optionally, a pH protective coating or layer; the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities; the barrier coating or layer comprising SiOx, wherein x is from 1.5 to 2.9, the barrier coating or layer being from 2 to 1000 nm thick, the barrier coating or layer having an outer surface facing the inner surfaces of the tie coating or layer and having an inner surface facing the cavities; and the pH protective coating or layer comprising SiOxCy or SiNxCy wherein x is from about 0.5 to about 2.4 and y is from about 0.6 to about 3, the pH protective coating or layer having an outer surface facing the inner surfaces of the barrier coating or layer and having an inner surface facing the cavities.

In some embodiments, the system useful in assessing the quality, purity, and integrity of the biopharmaceutical product during the manufacturing, packaging and delivery process comprises equipment for maintaining quality control over the biomanufacturing process, material, and products. Quality control (QC) and quality assurance (QA) are essential function of the biopharmaceutical industry, which require drug manufacturers to thoroughly test materials, processes, equipment, techniques, environments and personnel in order to ensure their final products are consistent, safe, effective, predictable, and free of defects. QC applications may include: microanalysis by FT-IR/Raman microscopy; microbial identification by MALDI-TOF-MS; determining compound identity and quantitation by NMR; raw material analysis by XRD spectrometry; counterfeit analysis by FT-IR spectroscopy; analysis of APIs by LC-MS. Examples of vessels that may be used for QC testing of the biopharmaceutical materials include microplates, microtiter plates, microwell plates, petri dishes, pipette tips, vials, and the like.

In some embodiments of the present disclosure, when aspects such as quality, quantity, purity, integrity, etc of biopharmaceutical products are tested, the contact surfaces of the equipment used to test these aspects of the intact cells would be identical to the contact surfaces of at least some of the vessels in which these cells were grown and harvested. In other embodiments, the contact surfaces of the equipment used to test these aspects of the intact cells would not be identical to the contact surfaces of the vessels in which these cells were grown and harvested. In some embodiments, when aspects such as quality, quantity, purity, integrity, etc of biopharmaceutical materials (i.e., the biopharmaceutical composition and/or its precursors) are tested, the contact surfaces of the equipment used to test these aspects of the biopharmaceutical would be identical to the contact surfaces of at least some of the vessels in the downstream process in which the biopharmaceutical product was purified, held, or packaged. In other embodiments, the contact surfaces of the equipment used to test these aspects of the biopharmaceutical product would be not identical to the contact surfaces of the vessels in the downstream process in which the biopharmaceutical product was purified, held, or packaged.

### Determining the Compatibility of the Provisional Contact Surface

An aspect of the disclosure is a method for manufacturing, packaging, or delivering a biopharmaceutical product, the method comprising: providing a biopharmaceutical product; providing a provisional contact surface; determining if the biopharmaceutical product is compatible with the provisional contact surface; modifying the provisional contact surface to increase its compatibility with the biopharmaceutical product, thereby producing a customized contact surface; and using the customized contact surface in two or more vessels or connectors, each vessel or connector comprising a cavity and an inner wall, wherein the inner wall of the two or more vessels comprises the customized contact surface in the manufacture, packaging, and delivery of the biopharmaceutical product.

In any embodiment, one or more of the following analytical test methods can be used for determining the compatibility of a provisional contact surface:

Radiolabeled Protein Adsorption experiments can be carried out according to previously published literature, for example the previously cited and incorporated articles authored by Mingchao Shen et al., Y. Vickie Pan et al., or T. A. Horbett. These methods are used in the working examples of this specification to determine surface adsorption and denaturing of peptides.

X-ray photo electron spectroscopy (XPS) can be carried out, for example, by using a PHI Quantum 2000 instrument sold by Physical Electronics, Eden Prairie, Minn. XPS can be used to determine the surface atomic composition, other than hydrogen, of plasma-treated microplates or other vessels and equipment. The X-ray source used can be a monochromated Alkα at 1486.6 eV. The acceptance angle and take-off angle can be ±23° and 45°, respectively. The analysis area can be 1400×300 μm. These conditions are used in the working examples of this specification to determine surface charge and the presence of hydrogen bond donor and acceptor groups.

Measurement of the water contact angle of a surface can be carried out, for example, by placing a 5-10 microliter droplet of distilled and deionized water on the surface to be evaluated. The angle formed between the vector at the solid liquid interface and the vector at the liquid-air interface is measured. These conditions are used in the working examples of this specification.

The measured water contact angle can be between 0 and 180 degrees. Generally, an angle greater than 90 degrees is considered hydrophobic and below 90 degrees is considered hydrophilic. An angle approaching 0 degrees is considered extremely hydrophilic and an angle approaching 180 degrees is considered extremely hydrophobic.

### Modifying the Provisional Contact Surface

In some embodiments of this aspect of the disclosure, the step of modifying or customizing the provisional contact surface enhances the compatibility of the provisional contact surface with the biopharmaceutical product, and includes at least one of the following steps. In some embodiments, the step of modifying the provisional contact surface is carried out at least in part by reducing the water contact angle of the provisional contact surface. In other embodiments, the step of modifying the provisional contact surface is carried out at least in part by reducing the percentage of H bond donor groups as determined by x-ray photoelectron spectroscopy (XPS) and hydrogen forward scattering (HFS) analysis of the provisional contact surface. In some embodiments, the step of modifying the provisional contact surface is carried out at least in part by reducing the percentage of H bond donor groups as determined by x-ray photoelectron spectroscopy (XPS) and hydrogen forward scattering (HFS) analysis or Rutherford backscattering (RBS) analysis of the provisional contact surface. Hydrogen forward scattering (HFS) analysis or Rutherford backscattering (RBS) analysis are two different analyses of hydrogen content of the contact surface. Either or both can be used. In other embodiments, the step of modifying the provisional contact surface is carried out at least in part by increasing the percentage of H bond donor groups as determined by x-ray photoelectron spectroscopy (XPS) and hydrogen forward scattering (HFS) analysis or Rutherford backscattering (RBS) analysis of the provisional contact surface. In some embodiments, the step of modifying the provisional contact surface is carried out at least in part by increasing the percentage of H bond donor groups as determined by x-ray photoelectron spectroscopy (XPS) and Rutherford backscattering (RBS) analysis of the provisional contact surface. In other embodiments, the step of modifying the provisional contact surface is carried out at least in part by reducing percentage of anion and cation groups or both as determined by XPS of the provisional contact surface. In yet other embodiments, the step of modifying the provisional contact surface is carried out at least in part by reducing the proportion of metal or metalloid atoms other than silicon, or both, of the provisional contact surface. The method is carried out by practicing any one or any combination of the steps in any order.

Another aspect of the disclosure is a customized biopharmaceutical product contact surface comprising the following statistical ratios of silicon, oxygen, carbon, and hydrogen atoms, in which the proportions of atoms other than hydrogen are determined by XPS and the proportions of hydrogen are determined by hydrogen forward scattering (HFS) or Rutherford backscattering (RBS): Si=1:O=x:C=y:H=z, in which x is from about 1.5 to about 2.9, y is from about 0 to about 1, and z is within the range from about 0 to about 4.

Optionally, the customized biopharmaceutical product contact surface has a water contact angle less than 90°. Optionally, the biopharmaceutical product contact surface has less than 1% H bond donor groups as determined by x-ray photoelectron spectroscopy (XPS) and hydrogen forward scattering (HFS) analysis. Optionally, the biopharmaceutical product contact surface has less than 1% H bond donor groups as determined by x-ray photoelectron spectroscopy (XPS) and Rutherford backscattering (RBS) analysis. Optionally, the biopharmaceutical product contact surface has at least 1% H bond acceptor groups as determined by XPS and HFS analysis. Optionally, the biopharmaceutical product contact surface has at least 1% H bond acceptor groups as determined by XPS and RBS analysis. Optionally, the customized biopharmaceutical product contact surface has less than 1% anion groups and cation groups as determined by XPS analysis. Optionally, the customized biopharmaceutical product contact surface is essentially free of metal or metalloid atoms other than silicon, as determined by XPS analysis.

Optionally in any embodiment, the customized contact surface can be applied to at least one of the following types of vessels to increase its compatibility with the particular biopharmaceutical product: 384 well plates; 96 well plates; bioreactors; blood sample collection tubes; bottles; bulk storage vessels; cannulas; capture columns; cartridges; catheters; cell bank vials; cell separators; cell vials; centrifugal pumps; centrifuge tubes; chromatography columns; chromatography vials; clarifiers; closures; container closure systems; cryopreservation vessels; culture bottles; delivery containers; diafiltration equipment; dispensing units, ELISA plates; elution bags; evacuated blood sample collection tubes; fill/finish equipment; filtration equipment; freeze dryer equipment; harvest vessels; shakers; in-process analysis instruments; intermediate columns; intravenous (IV) bags; media bags; media bottles; media vessels; membrane chromatography columns; microplates; microtiter plates; microwell plates; mixing bags; monitoring devices; multiple-use bioreactors; package filling apparatus; pumps; petri dishes; pipette tips; plates; plungers; polish columns; pre-filled syringes; prefilled syringe with luer lock fittings; primary packaging; production bioreactors; production fermenters; roller bottles; sample collection tubes; sampling vessels; seed fermenters; separators; shake flasks; single-use bioreactors; slides; spinner flasks; staked needle pre-filled syringes; storage bags; sterilizers; stock culture vials; storage containers; syringes (prefilled); tanks; terminal reactors; transfer bags; ultrafiltration/diafiltration equipment; ultrafiltration equipment; upstream bioreactors; valves; vials; viral filtration equipment; viral inactivation vessels; and wave bioreactors. Optionally in any embodiment, the modified contact surface can be applied to multiple (2, 3, 4, etc) types of vessels listed in the preceding list. Optionally in any embodiment, the customized contact surface can be specified for use on the contact surfaces of at least two vessels or equipment coming in contact with the biopharmaceutical product during its manufacture, packaging, or delivery.

### Characteristics of a Surface Resisting Non-Specific Protein Adsorption

In other embodiments of this disclosure, the contact surfaces, and preferably two or more, and more preferably, more than two, are customized for a given biopharmaceutical product so as to resist or reduce non-specific protein adsorption, for example, by customizing silica-based coatings applied by plasma enhanced chemical vapor deposition (“PECVD”): (1) a hydrophilic/polar surface, (2) a surface having a substantial proportion of hydrogen-bond acceptors, (3) a surface not having a substantial proportion of hydrogen-bond donors, and (4) an uncharged surface.

The composition of the coatings on the contact surfaces can be tailored by adjusting the ratio of organosilicon gas to oxygen gas flowrate mixture and the applied voltage or power of the PECVD process. A wide variety of coatings can be produced with different bulk and surface properties. For example, pure and high-density silica (i.e. SiO₂) can be deposited with excellent gas and leachable barrier properties and hydrophilic surface properties. This is accomplished with a high oxygen to organosilicon gas ratio in the gas mixture and high applied power. All the carbon from the organosilicon gas is removed from the process by the vacuum system as COₓ gas, which is a byproduct of the reaction between organosilicon gas and oxygen.

### Hydrophilic/Polar Surface

In other aspects, the silica-based coatings can be optimized to provide a suitable water contact angle for contact with a biopharmaceutical product, for example, a contact angle of 30-60 degrees, which is moderately hydrophilic.

Alternatively, the coatings can be made more hydrophilic during PECVD by maintaining a high oxygen to organosilicon ratio, but reduced power. Water is a byproduct of the reaction between the organosilicon compound and oxygen. At high power, water vapor is removed from the vacuum system. At reduced power, water vapor can be incorporated into the coating as silanol (i.e. Si—OH) groups. Silanol groups are polar, which increases the surface hydrophilicity. Carried to an extreme, a water contact angle near zero can be provided.

In other aspects, a more hydrophobic surface can be produced by reducing the oxygen to organosilicon flowrate ratio at moderate to low power. This incorporates non-polar aliphatic or other carbon-and-hydrogen-containing groups into the surface and thus reducing hydrophilicity or increasing hydrophobicity. The resulting coating is better characterized as an organosilica or organosiloxane because it contains a substantial proportion of carbon atoms, as well as silicon and oxygen atoms, through its molecular structure. Alternatively, a water contact angle of 80-100 degrees can be achieved on an organosiloxane coating. Further hydrophobicity can be incorporated by reducing polar groups and increasing surface roughness. This can be accomplished by eliminating oxygen from the gas mixture and even selecting siloxane monomers with a higher carbon to oxygen ratio. Silane monomers that have no oxygen in their structure, such as tetramethylsilane (SiC₄H₁₂), and trimethylsilane (SiC₃H₁₀), can produce very hydrophobic coatings. Water contact angles over 100 degrees are possible in this case.

### Hydrogen-bond Acceptors and Donors

In other aspects, these silica-based coatings can be optimized to provide a coating that has fewer hydrogen bond donors and more hydrogen bond acceptors.

Hydrogen-bond donor groups contain a highly electronegative atoms (e.g. O, N, Cl, F) covalently bonded to a hydrogen atom. Hydrogen bond acceptor groups contain a highly electronegative atom that has a lone pair of electrons. The hydrogen bond is formed between a hydrogen bond donor group and acceptor group. The hydrogen bond is weaker than covalent and ionic bonds, but stronger than Van Der Waals bonds. Examples of hydrogen bond acceptors and donors in organic compounds generally are shown in Table 1.

TABLE 1
List of chemical bonds types as hydrogen bond donors and/or acceptors.

| Bond Type | Formula | Acceptor | Donor |
|---|---|---|---|
| Carbonyl | C═O | YES | |
| Amine | —NH2 | YES | YES |
| Amide | 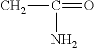 | YES | YES |
| Ester | 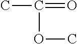 | YES | |
| Ether | C—O—C | YES | |
| Aldehyde | 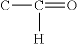 | YES | YES |
| Ketone | 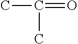 | YES | |
| Alcohol | CO—H | YES | YES |
| Silanol | SiO—H | YES | YES |
| Carboxyl | O═CO—H | YES | YES |
| Aliphatic | CH₃—CH₂—CH₃ | NO | NO |
| Water | H₂O | YES | YES |

Bond types that are particularly useful in a contact surface for many biopharmaceutical products are hydrogen bond acceptors and are not hydrogen bond donors.

### Uncharged Surface

Some biopharmaceutical products have a charged surface, and such products tend to be denatured by and adsorbed on a charged contact surface. Consequently, customizing the contact surface generally is preferred in those situations.

### EXAMPLES

All examples are based on silica-based coatings deposited by plasma enhanced chemical vapor deposition (PECVD). The precursor utilized during PECVD for all examples is hexamethyldisiloxane (HMDSO) mixed with oxygen gas. Alternative precursors within the siloxane chemical family could be used similarly. The coatings could be deposited on any vessels or equipment useful in the discovery, development, manufacture, packaging and delivery of biopharmaceutical products. In all cases the silica-based coating is in direct contact with the biopharmaceutical products.

Examples 1-3 provide preferred silica-based coatings that have utility for minimizing biologic (i.e. protein) drug adsorption, denaturing and aggregation compared to ordinary borosilicate glass or ordinary plastics. Example 4 provides the more preferred silica based coatings.

Achieving the favorable surface characteristics of silica-based coatings by PECVD that reduce or prevent protein adsorption, denaturing and aggregation are accomplished by adjusting the energy density of the plasma. The energy density of the plasma can be defined by a composite parameter W/FM, which is in units of Joules per kilogram or J/kg. W corresponds to the applied power in Watts, F is the flowrate in standard cubic centimeters per minute or sccm and M is the molecular weight of the plasma polymerizing components of the gas mixture. The following equation is used to calculate W/FM of a mixture of HMDSO and oxygen gas in units of J/kg:

$$\frac{W}{[ {F}_{H M D S O} {M}_{H M D S O} + {F}_{o x y g e n} {M}_{o x y g e n} ]} \times 1.34 \times 1 {0}^{9}$$

TABLE 2
PECVD process conditions and composite parameter
W/FM that correspond to the various silica-
based coatings in Examples 1-4.

| | | HMDSO | Oxygen | | |
|---|---|---|---|---|---|
| | | Flowrate | Flowrate | Power | W/FM |
| Coating | Example | (sccm) | (sccm) | (W) | (kJ/kg) |
| SiO₂ | 1 | 3 | 45 | 500 | 348 |
| SiOₓC_{y}H_{z} | 2 | 10 | 10 | 500 | 276 |
| SiOₓH_{y} | 3 | 6 | 200 | 200 | 36 |
| SiOₓC_{y}H_{z} | 4 | 10 | 40 | 500 | 185 |

Note: Molecular weight of HMDSO is 162.4 g/mol. Molecular weight of 02 is 32 g/mol.
The chemical reactions that correspond to the different coatings summarized in Table 1:

1) SiO₂C₆H₁₈+2½O₂→SiO₂+9H₂O+6CO₂; Example 1

2) SiO₂C₆H₁₈+AO₂→BSiOₓH_{z}+CH₂O+DCO₂; Example 3

3) SiO₂C₆H₁₈+AO₂→BSiOₓC_{y}H_{z}+CH₂O+DCO₂; Examples 2, 4

TABLE 3
Typical chemical functional groups found in silica-based coatings.

| | | Affinity for | Hydrogen bond | | |
|---|---|---|---|---|---|
| # | Bond | Water | donor/acceptor | Charge | Example |
| 1 | Si—O—Si | Hydrophobic | Neither | Neutral | 1, 2, 3 |
| 2 | Si—(CH₃)ₓ | Hydrophobic | Neither | Neutral | 2 |
| 3 | Si—CH₂ | Hydrophobic | Neither | Neutral | 2 |
| 4 | Si—OH | Hydrophilic | Donor | Neutral | 2, 3 |
| 5 | Si—H | Hydrophobic | Neither | Neutral | 3 |
| 6 | C—H | Hydrophobic | Neither | Neutral | 2 |

### Example 1

A preferred contact surface with chemical characteristics that reduce interactions that lead to denaturing and aggregation of a biopharmaceutical product is described below. The silica-based coating is pure silicon dioxide (i.e. SiO₂) with the following chemical functional group characteristics (Table 4):
Net Charge Neutral (No anion or cation groups detected by XPS; <1%)
Hydrophilic (Water contact angle 30-80 degrees)
Hydrogen bond donor groups (As detected by XPS; <1%)
Absence of hydrogen bond acceptor groups (As detected by XPS; <1%)
Absence of all aliphatic groups (As detected by XPS; <1%) Absence of alkali, alkaline, transition, metalloids (with the exception of Si) and post-transition metals.

These characteristics are favorable for reducing the adsorption of more hydrophobic and charged proteins and peptides in a biopharmaceutical product as determined by radiolabeled protein adsorption testing. Adsorption is expected to be less than 30 ng/cm².

### Example 2

A preferred contact surface with chemical characteristics that reduce interactions that lead to adsorption, denaturing and aggregation of a biopharmaceutical product is described below. The silica-based coating is organosiloxane (i.e. SiOₓC_{y}H_{z}) with the following chemical functional group characteristics (Table 4):
Net Charge Neutral (No anion or cation groups detected by XPS; <1%)
Hydrophilic (Water contact angle 50-90 degrees)
Hydrogen bond donor groups (As detected by XPS; 1-10%)
Absence of hydrogen bond acceptor groups (As detected by XPS; <1%)
Absence of alkali, alkaline, transition, metalloids (with the exception of Si) and post-transition metals.

These characteristics are favorable for reducing the adsorption of more hydrophilic and charged proteins and peptides in a biopharmaceutical product as determined by radiolabeled protein adsorption testing. Adsorption is expected to be less than 30 ng/cm².

### Example 3

A preferred contact surface with chemical characteristics that reduce interactions that lead to denaturing and aggregation of a biopharmaceutical product is described below. The silica-based coating is a silicone oxide (i.e. SiOₓH_{y}) with the following chemical functional group characteristics (Table 4):
Net Charge Neutral (No anion or cation groups detected by XPS; <1%)
Hydrophilic (Water contact angle <50 degrees)
Hydrogen bond donor groups (Si—OH; As detected by XPS; 1-50%)
Absence of hydrogen bond acceptor groups (As detected by XPS; <1%)
Absence of alkali, alkaline, transition, metalloids (with the exception of Si) and post-transition metals.

These characteristics are favorable for reducing the adsorption of more hydrophilic and charged proteins and peptides in a biopharmaceutical product as determined by radiolabeled protein adsorption testing. Adsorption is expected to be less than 30 ng/cm².

### Example 4

Another preferred contact surface with chemical characteristics that reduce interactions that lead to denaturing and aggregation of a biopharmaceutical product is described below. The silica-based coating is a silicone oxide (i.e. SiOₓC_{y}H_{z}) with the following chemical functional group characteristics (Table 4):
Net Charge Neutral (No anion or cation groups detected by XPS; <1%)
Hydrophilic (Water contact angle <50 degrees)
Absence of hydrogen bond donor groups (Si—OH; As detected by XPS; <1%)
Hydrogen bond acceptor groups (As detected by XPS; 1-50%)
Absence of alkali, alkaline, transition, metalloids (with the exception of Si) and post-transition metals.

These characteristics are favorable for reducing the adsorption of more hydrophilic and charged proteins and peptides in a biopharmaceutical product as determined by radiolabeled protein adsorption testing. Adsorption is expected to be less than 30 ng/cm².

TABLE 4
Table 4. Chemical functional group characteristics and expected peptide reactivity

| | | | | | | | | | | | | Expected Peptide | |
|---|---|---|---|---|---|---|---|---|---|---|---|---|---|
| | | | | | | Expected surface properties | | | | | Metals | Receptivity | |
| | | Coating Conditions | | | | | H Bond | H Bond | Organic | Charge | and | | Expected |
| Sample | | HMDSO | Oxygen | | | | Donor | Acceptor | Carbon | (anions, | Metal- | | value as |
| | Drug | Flow- | Flow- | | | Water | Groups | Groups | groups | cations, | loids | Expected | peptide |
| Exam- | Contact | rate | rate | Power | W/FM | contact | (by | (by | (by | by | (other | adsorption | contact |
| ple | Surface | (sccm) | (sccm) | (W) | (kJ/kg) | angle | XPS) | XPS) | XPS) | XPS) | than Si) | (ng/cm²) | surface |
| 1 | SiO₂ | 3 | 45 | 500 | 348 | 30-80° | 1-50% | <1% | <1% | <1% | Absent | <30 | preferred |
| 2 | SiOx Hz | 6 | 200 | 200 | 36 | <50° | 1-50% | <1% | <1% | <1% | Absent | <30 | preferred |
| 3 | SiOxCyHz | 10 | 10 | 500 | 276 | 50-90° | 1-10% | <1% | 30-50 | <1% | Absent | <30 | preferred |
| 4 | SiOxCyHz | 10 | 40 | 500 | 185 | <50° | <1% | 1-50% | 20-40 | <1% | Absent | <30 | most |
| | | | | | | | | | | | | | preferred |

### Example 5

Protein adsorption on the surface of vessels for parenteral administration of biopharmaceutical products, particularly proteins and antibodies, has been implicated as the precursor to protein aggregation. Aggregate formation of these products in formulations and subsequent complement activation, can trigger adverse drug interactions (ADR) in subjects to whom the biopharmaceutical product is administered. Proteins denature from the formulation by surface adsorption then slough off as aggregates. Systems used in the manufacture and packaging of biopharmaceutical products, such as antibodies and recombinant proteins, should ideally comprise contact surfaces which minimize protein adsorption, reduce protein aggregate formation and ultimately the risk of ADRs.

IgG protein adsorption and retention on vessels (e.g., tubes) whose surfaces are characterized by the contact surfaces of this disclosure were compared to that of uncoated polymer cyclic olefin polymer (COP) containers. The coatings in the polymer tubes are composed of proprietary silica (bilayer) and organosilica (trilayer) coatings of the disclosure. The various reagents for the following studies described are as follows: Sodium azide (Sigma-Aldrich, MO); Sodium iodide (NaI) (Sigma-Aldrich, MO); Sodium phosphate monobasic (Fisher Scientific, NJ); Sodium hydroxide (Fisher Scientific, NJ); Sodium chloride crystals (NaCl) (EMD Millipore, MA); and Citric acid, monohydrate (J.T. Baker, NJ).

### IgG Radiolabeling, Purification and Collection

Bovine IgG (Sigma-Aldrich, MO) was radiolabeled using the iodine monochloride (ICl) method as modified in Horbett T. A. J. Biomed. Mater. Res. (1981) 15(5):673-695. Briefly, 1 mCi of Iodine-125 radionuclide (Perkin-Elmer, MA) was added to 0.5 ml 2× boric acid solution (Fisher Scientific, NJ), to which 0.5 ml of ICl/NaCl mixture in a 2:1 ratio was added. Next, 0.5 ml of a 10 mg/ml bovine IgG in a citrate phosphate buffered saline solution with sodium azide (cPBSz) was added and the iodination reaction was performed on ice for 20 mins and then run through a size exclusion chromatography column. 40 fractions were collected to capture the labeled protein and free iodine peaks to evaluate iodination efficiency. The fractions from protein peaks were pooled together and run through a second chromatography column, repeating fraction collection and peak identification. Purified labeled protein fractions were pooled together, placed in a lead vessel and frozen in −80° C. freezer until further use.

### Protein Adsorption Study

Prior to the protein adsorption study, treated tubes (n=5 per group of bilayer coated tubes, trilayer coated tubes, and uncoated COP tubes) were soaked in 1 ml cPBS solution with 10 mM NaI (cPBSzI) for 1 hr. For the adsorption, the labeled IgG was thawed and then added to a 0.1 mg/mL solution of bovine IgG in cPBSzI to create radiolabeled bovine IgG or “hot” protein. The cPBSzI buffer was aspirated with a transfer pipette and 1 mL of the “hot” protein was then added to each tube, and allowed to adsorb for 2 hrs before rinsing three times with cPBSzI. The radioactivity of each rinsed tube was then measured for 1 min along with protein standards using a Perkin Elmer Wizard 2 Gamma Counter.

### Protein Retention Study

In order to assess protein retention in the tubes, i.e., how tightly retained the protein is on the surface of the tubes, “hot” IgG protein was first adsorbed for 2 hrs in the tubes (n=10 per group) as described previously. Next, 1 ml of 1% SDS solution was added to each of the tubes and allowed to sit overnight in order to extract the IgG protein. The SDS solution was aspirated using a transfer pipette and the tubes were rinsed again with cPBSzI three times, and their radioactivity was measured for 1 min along with protein standards using a Perkin Elmer Wizard 2 Gamma Counter. Untreated tubes were used as controls. Adsorption and retention data was reported in ng/cm².

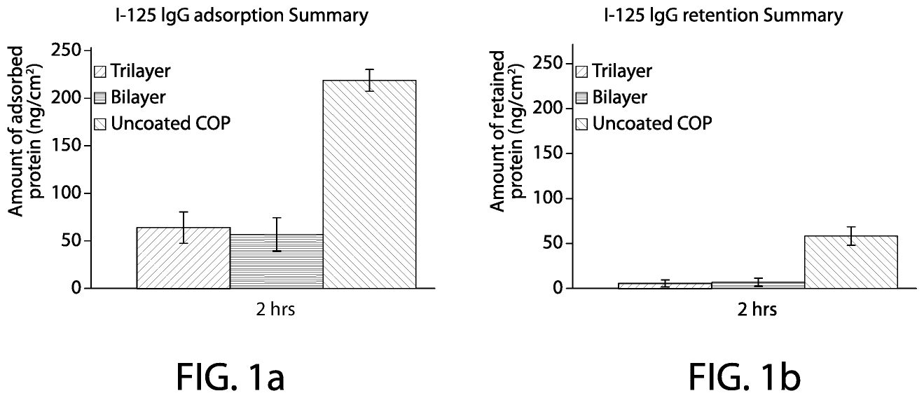

As shown in FIG. 1a, tubes with the bilayer and trilayer coatings exhibit 4-5 times less IgG protein adsorption compared to the uncoated COP tubes. As shown in FIG. 1b, SDS extraction can remove adsorbed IgG protein from all tubes. However, a higher amount of adsorbed IgG protein is retained on the surface of uncoated COP tubes, compared with that retained in tubes with the bilayer or trilayer coatings. Finally, a higher amount of adsorbed protein was removed from the uncoated COP tubes after rinsing compared to the coated vessels.

Results: These results demonstrate that systems of the present disclosure with contact surfaces comprising bilayer or trilayer coatings provide compatible surfaces that minimize protein adsorption and retention on the surface of the coated vessels.

1. A system comprising:
at least two vessels capable of use in the manufacture, packaging, or delivery of a biopharmaceutical product, wherein
each of said vessels comprises walls defining inner cavities, the vessels being optionally open or capable of being opened on one end or on one side;
the walls of each of said vessels having inner surfaces facing the cavities, the inner surfaces being characterized by a common surface residing thereon in substantially all areas where the inner surfaces are in contact with the biopharmaceutical product, and
the common surface comprising:
a tie coating or layer;
a barrier coating or layer; and
optionally, a pH protective coating or layer;
wherein the tie coating or layer comprising SiOₓC_{y} or SiNₓC_{y} wherein x is from about 0.5 to about 2.4 and y is from about 0.6 to about 3, the tie coating or layer having an outer surface facing the walls of the vessels and having an inner surface facing the cavities;
wherein the barrier coating or layer comprising SiOₓ, wherein x is from 1.5 to 2.9, the barrier coating or layer being from 2 to 1000 nm thick, the barrier coating or layer having an outer surface facing the inner surface of the tie coating or layer and having an inner surface facing the cavities, and
wherein the pH protective coating or layer comprising SiOₓC_{y} or SiNₓC_{y} wherein x is from about 0.5 to about 2.4 and y is from about 0.6 to about 3, the pH protective coating or layer having an outer surface facing the inner surface of the barrier coating or layer and having an inner surface facing the cavities.

2. The system of claim 1, wherein each of the vessels from that least two vessels is selected from a group consisting of one or more of the following: 384 well plates; 96 well plates; bioreactors; blood sample collection tubes; bottles; bulk storage vessels; cannulas; capture columns; cartridges; catheters; cell bank vials; cell separators; cell vials; centrifugal pumps; centrifuge tubes; chromatography columns; chromatography vials; clarifiers; closures; container closure systems; cryopreservation vessels; culture bottles; delivery containers; diafiltration equipment; dispensing units, ELISA plates; elution bags; evacuated blood sample collection tubes; fill/finish equipment; filtration equipment; freeze dryer equipment; harvest vessels; shakers; in-process analysis instruments; intermediate columns; intravenous (IV) bags; media bags; media bottles; media vessels; membrane chromatography columns; microplates; microtiter plates; microwell plates; mixing bags; monitoring devices; multiple-use bioreactors; package filling apparatus; pumps; petri dishes; pipette tips; plates; plungers; polish columns; pre-filled syringes; pre-filled syringe with luer lock fittings; primary packaging; production bioreactors; production fermenters; roller bottles; sample collection tubes; sampling vessels; seed fermenters; separators; shake flasks; single-use bioreactors; slides; spinner flasks; staked needle pre-filled syringes; storage bags; sterilizers; stock culture vials; storage containers; tanks; terminal reactors; transfer bags; ultrafiltration/diafiltration equipment; ultrafiltration equipment; upstream bioreactors; valves; vials; viral filtration equipment; viral inactivation vessels; and wave bioreactors.

3. The system of claim 1, wherein at least one of the vessels is selected from a group consisting of bioreactors, pumps, cell bank vials, harvest vessels, stock culture vials, fermenters, shake flasks, flasks, shakers, columns, separators, bags, beakers, tanks, cylinders, bulk storage units or containers, single use bags, roller bottles, storage tubes, sampling vessels, syringes, dispensing units, pipette tips, slides, vials and intravenous (IV) bags.

4. (canceled)

5. The system of claim 1, wherein the at least two vessels comprise;
(a) a first vessel useful for holding a bulk amount of the biopharmaceutical product; and
(b) a second vessel useful for administering a composition comprising at least a portion of said biopharmaceutical product to a subject in need thereof.

6. The system of claim 1, wherein the at least two vessels further comprise one or more connectors connecting one or more of the vessels to one or more of another vessel or connector, wherein at least one of said connectors has a wall whose inner surface is characterized by a surface residing thereon in substantially all the areas that come into contact with the biopharmaceutical product during its production, the surface being common to the innermost surface of the one or more of the vessels and the one or more of the another vessels or connector, and wherein at least one of the connectors is independently selected from a group consisting of tubes, tubings, valves, and pipes.

7. (canceled)

8. (canceled)

9. (canceled)

10. The system of claim 5, further comprising one or more series of vessels useful in the production of said bulk amount of the biopharmaceutical product,
wherein at least one of said vessels in the one or more series has a wall whose inner surface is characterized by a surface residing thereon in substantially all the areas that come into contact with the biopharmaceutical product during its production, the surface being not common to that of the first and second vessels, and
wherein the surface residing on said wall in substantially all the areas that come into contact with the biopharmaceutical product during its production, is common to the surface on the wall of at least one of another of said vessels in the one or more series.

11. The system of claim 10, wherein the common surface comprises the tie coating or layer, the barrier coating or layer, and optionally, t pH protective coating or layer;
wherein the tie coating or layer comprising SiOₓC_{y} or SiNₓC_{y} wherein x is from about 0.5 to about 2.4 and y is from about 0.6 to about 3, the tie coating or layer having an outer surface facing the walls of the vessels and having an inner surface facing the cavities;
wherein the barrier coating or layer comprising SiOₓ, wherein x is from 1.5 to 2.9, the barrier coating or layer being from 2 to 1000 nm thick, the barrier coating or layer having an outer surface facing the inner surface of the tie coating or layer and having an inner surface facing the cavities; and
wherein the pH protective coating or layer comprising SiOₓC_{y} or SiNₓC_{y} wherein x is from about 0.5 to about 2.4 and y is from about 0.6 to about 3, the pH protective coating or layer having an outer surface facing the inner surface of the barrier coating or layer and having an inner surface facing the cavities.

12. A system comprising
at least two vessels capable of use in assessing one or more of the following characteristics: quality, purity, and integrity of one or more of a biopharmaceutical product, wherein
each of said vessels comprises walls defining inner cavities, the vessels being optionally open or capable of being opened on one end or on one side;
the walls of each of said vessels having inner surfaces facing the cavities, the inner surfaces being characterized by common surfaces residing thereon in substantially all areas where the inner surfaces are in contact with the biopharmaceutical product, and the common surface comprising
a tie coating or layer;
a barrier coating or layer; and
optionally, a pH protective coating or layer;
wherein the tie coating or layer comprising SiOxCy or SiNxCy wherein x is from about 0.5 to about 2.4 and y is from about 0.6 to about 3, the tie coating or layer having an outer surface facing the walls of the vessels and having an inner surface facing the cavities;
wherein the barrier coating or layer comprising SiOx, wherein x is from 1.5 to 2.9, the barrier coating or layer being from 2 to 1000 nm thick, the barrier coating or layer having an outer surface facing the inner surfaces of the tie coating or layer and having an inner surface facing the cavities; and
wherein the pH protective coating or layer comprising SiOxCy or SiNxCy wherein x is from about 0.5 to about 2.4 and y is from about 0.6 to about 3, the pH protective coating or layer having an outer surface facing the inner surfaces of the barrier coating or layer and having an inner surface facing the cavities.

13. The system of claim 30, wherein the common surface is either identical or not identical to one or both of a common surface of a first vessel useful for holding a bulk amount of the biopharmaceutical product and a second vessel useful for administering a composition comprising at least a portion of said biopharmaceutical product to a subject in need thereof, and to the common surface of the one or more connectors.

14. (canceled)

15. The system of claim 12, wherein each vessel is selected from a group consisting of microplates, microtiter plates, microwell plates, and petri dishes.

16. The system of claim 1, wherein the biopharmaceutical product is selected from the group consisting of one of more of the following: an antibody, a bulk biopharmaceutical formulation, a cell culture formulation, a cell suspension, a culture fluid, a biopharmaceutical product, a biopharmaceutical substance, a biopharmaceutical formulation, an expression vector/host formulation, a harvested cell formulation, a host cell composition, a host cell contaminant, a mobile phase, a monoclonal antibody formulation, a product stream, a seed train composition, a stationary phase, a peptide or protein formulation, and a nucleic acid formulation.

17. A method for producing a system comprising at least two vessels or connectors useful in the manufacture, packaging, or delivery of a biopharmaceutical product, each of said vessels or connectors comprising walls defining inner cavities, the vessels being optionally open or capable of being opened on one end or on one side; the walls of each of said vessels or connectors having inner surfaces facing the cavities, the method comprising:
providing a biopharmaceutical product;
providing a provisional contact surface;
determining if the biopharmaceutical product is compatible with the provisional contact surface;
modifying the provisional contact surface to improve its compatibility with the biopharmaceutical product, thereby producing a customized contact surface; and
using the customized contact surface to produce the system such that the inner surfaces of the vessels/connectors are characterized by the customized common surface residing thereon in substantially all areas where the inner surfaces are in contact with the biopharmaceutical product, the customized common surface comprising
a tie coating or layer;
a barrier coating or layer; and
optionally, a pH protective coating or layer;
wherein the tie coating or layer comprising SiOₓC_{y} or SiNₓC_{y} wherein x is from about 0.5 to about 2.4 and y is from about 0.6 to about 3, the tie coating or layer having an outer surface facing the walls of the vessels and having an inner surface facing the cavities;
wherein the barrier coating or layer comprising SiOₓ, wherein x is from 1.5 to 2.9, the barrier coating or layer being from 2 to 1000 nm thick, the barrier coating or layer having an outer surface facing the inner surface of the tie coating or layer and having an inner surface facing the cavities; and
wherein the pH protective coating or layer comprising SiOₓC_{y} or SiNₓC_{y} wherein x is from about 0.5 to about 2.4 and y is from about 0.6 to about 3, the pH protective coating or layer having an outer surface facing the inner surface of the barrier coating or layer and having an inner surface facing the cavities.

18. The method of claim 17, wherein the biopharmaceutical product is selected from the group consisting of one of more of the following: an antibody, a bulk biopharmaceutical formulation, a cell culture formulation, a cell suspension, a culture fluid, a biopharmaceutical product, a biopharmaceutical substance, a biopharmaceutical formulation, an expression vector/host formulation, a harvested cell formulation, a host cell composition, a host cell contaminant, a mobile phase, a monoclonal antibody formulation, a product stream, a seed train composition, a stationary phase, a peptide or protein formulation, and a nucleic acid formulation.

19. The method of claim 17 where in the provisional contact surface has a water contact angle of less than 90°.

20. The method of claim 17, wherein provisional contact surface is characterized by one or more of:
having less than or at least 1% H bond donor groups as determined by x-ray photoelectron spectroscopy (XPS) and optionally by hydrogen forward scattering (HFS) analysis or Rutherford backscattering (RBS) analysis,
having less than 1% anion and cation groups as determined by XPS,
being essentially free of metal or metalloid atoms other than silicon as determined by XPS, and
being a plasma-enhanced chemical vapor deposition (PECVD) coating.

21. (canceled)

22. (canceled)

23. (canceled)

24. (canceled)

25. The method of claim 17, wherein the provisional contact surface has the following statistical ratios of silicon, oxygen, carbon, and hydrogen atoms, as determined by XPS and optionally by HFS or RBS: Si=1:O=x:C=y:H=z, in which x is from about 0.5 to about 2.9, y is from about 0 to about 3, and z is within the range from about 0 to about 9.

26. (canceled)

27. The method of claim 17, wherein the provisional contact surface has the following statistical ratios of silicon, oxygen, carbon, and hydrogen atoms, determined by XPS and optionally by HFS or RBS: Si=1:O=x:C=y:H=z, in which x is from 1.5 to 2.9, y is about 0, and z is about 0.

28. The method of claim 17, wherein the vessel is selected from a group consisting of one or more of the following: 384 well plates; 96 well plates; bioreactors; blood sample collection tubes; bottles; bulk storage vessels; cannulas; capture columns; cartridges; catheters; cell bank vials; cell separators; cell vials; centrifugal pumps; centrifuge tubes; chromatography columns; chromatography vials; clarifiers; closures; container closure systems; cryopreservation vessels; culture bottles; delivery containers; diafiltration equipment; dispensing units, ELISA plates; elution bags; evacuated blood sample collection tubes; fill/finish equipment; filtration equipment; freeze dryer equipment; harvest vessels; shakers; in-process analysis instruments; intermediate columns; intravenous (IV) bags; media bags; media bottles; media vessels; membrane chromatography columns; microplates; microtiter plates; microwell plates; mixing bags; monitoring devices; multiple-use bioreactors; package filling apparatus; pumps; petri dishes; pipette tips; plates; plungers; polish columns; pre-filled syringes; prefilled syringe with luer lock fittings; primary packaging; production bioreactors; production fermenters; roller bottles; sample collection tubes; sampling vessels; seed fermenters; separators; shake flasks; single-use bioreactors; slides; spinner flasks; staked needle pre-filled syringes; storage bags; sterilizers; stock culture vials; storage containers; syringes (prefilled); tanks; terminal reactors; transfer bags; ultrafiltration/diafiltration equipment; ultrafiltration equipment; upstream bioreactors; valves; vials; viral filtration equipment; viral inactivation vessels; and wave bioreactors.

29. The method of claim 17, wherein the method of modifying the provisional contact surface of a vessel to increase its compatibility with a particular biopharmaceutical product to produce a customized contact surface comprises at least one of the following steps:
reducing the water contact angle of the provisional contact surface by about 10%-90% of the original water contact angle,
reducing the percentage of H bond donor groups of the provisional contact surface by about 10%-90%, as determined by XPS analysis and optionally HFS analysis or RBS analysis;
increasing the percentage of H bond acceptor groups of the provisional contact surface by about 10%-90%, as determined by XPS analysis and optionally HFS analysis or RBS analysis;
reducing the percentage of anion groups, cation groups, or both, of the provisional contact surface by about 10%-90%, as determined by XPS analysis; and
reducing the percentage of metal or metalloid atoms, other than silicon, of the provisional contact surface by about 10%-90%, as determined by XPS.

30. The system of claim 12, wherein the at least two vessels further comprise one or more connectors connecting one or more of the vessels to one or more of another vessel or connector, wherein at least one of said connectors has a wall whose inner surface is characterized by a surface residing thereon in substantially all the areas that come into contact with the biopharmaceutical product during its production, the surface being common to the innermost surface of the one or more of the vessels and the one or more of the another vessel or connector.
